(19) United States
(12) Patent Application Publication
(10) Pub. No.: US 2005/0048578 A1
(43) Pub. Date: Mar. 3, 2005

(54) Methods of screening for monoclonal antibodies with desirable activity

(75) Inventor: Dongxiao Zhang, Moraga, CA (US)

Correspondence Address: BOZICEVIC, FIELD & FRANCIS LLP, 1900 UNIVERSITY AVE, SUITE 200, EAST PALO ALTO, CA, 94303, US

(73) Assignee: Epitomics, Inc.

(21) Appl. No.: 10/869,355

(22) Filed: Jun. 15, 2004

Related U.S. Application Data
(60) Provisional application No. 60/484,185, filed on Jun. 30, 2003.
(60) Provisional application No. 60/483,391, filed on Jun. 26, 2003.

(51) Int. Cl. G01N 33/53; C12N 5/06; C12N 15/85

(52) U.S. Cl. 435/7.1; 435/69.1; 435/455; 435/326; 530/387.1

(57) ABSTRACT

The invention provides methods for screening for a monoclonal antibody with a desirable activity. The methods involve altering a nucleic acid encoding a selected parental humanized monoclonal antibody to make a library of nucleic acids, introducing the library into mammalian cells such that a library of monoclonal antibodies are produced on the surfaces of the mammalian cells, and sorting the cells to isolate a cell producing a humanized monoclonal antibody with a desirable activity, e.g., increased affinity for an binding partner as compared to the parental antibody. This isolated cell may be cultured and used to reiterate the method. The subject methods find use in a variety of different industrial, medical and research applications.

### FIELD OF THE INVENTION

The field of this invention is monoclonal antibodies. The invention relates to methods of screening for a monoclonal antibody with a desirable activity, e.g., a binding activity.

### BACKGROUND OF THE INVENTION

Affinity maturation is a process of variation and selection and involves a subset of lymphocytes, B-cells, as they mature in the bone marrow. When a B-cell is activated by binding to an antigen it firstly secretes soluble antibodies that bind to the antigen, and secondly it divides to clone itself. This cloning process is done with a very high mutation rate, called somatic hypermutation, which results in daughter B-cells that have mutated immunoglobulin genes that encode antibodies that are different from the parent. These daughter B-cells also, in turn, are activated and cloned if they bind to the antigen. The higher the affinity of a B-cell for antigen present, the more likely it is that the B-cell will divide to clone itself, resulting in a population of B-cells that produce antibodies with a higher affinity to the antigen than the antibodies of the parental B-cell. Affinity maturation is therefore an in vivo process by which, using a single low affinity parental antibody as starting material, a population of high affinity antibodies is generated.

The conventional method for the production of monoclonal antibodies involves hybridomas (Köhler & Milstein, Nature 256:495-7, 1975). In this method, splenic or lymphocyte cells from a mammal which has been injected with antigen are fused with a tumor cell line, thus producing hybrid cells. These hybrid cells, or “hybridomas”, are both immortal and capable of producing an encoded antibody. To select a hybridoma producing a single antibody, the hybridomas made by cell fusion are segregated by selection, dilution, and regrowth until a single genetically pure antibody-expressing cell line is selected. Because hybridomas produce homogeneous antibodies against a desired antigen, they are called “monoclonal” antibodies. Hybridoma technology has primarily been focused on the fusion of murine lines, but also human-human hybridomas, human-murine hybridomas, rabbit-rabbit hybridomas and other xenogenic hybrid combinations have been made.

Monoclonal antibodies produced by hybridomas, because they are made using splenic or lymphocyte cells, have not yet undergone affinity maturation, and, as such, have yet to achieve their maximum affinity for an antigen. As such, many antibodies made using hybridoma technology may have low antigen affinity for an antigen, but have the potential to be modified to increase the its binding affinity.

While many strategies have been proposed for mimicking affinity maturation in vitro, they usually involve antibodies that are expressed in bacterial, bacteriophage or yeast systems. However, since antibodies are glycosylated in mammalian cells, and glycosylation can affect the binding activities of an antibody, it is desirable to mimic affinity maturation in mammalian cells.

As such, there is a need for improved methods for increasing the affinity of an antibody, particularly an antibody produced in mammalian cells. The present invention addresses this, and other, needs.

Literature

Literature of interest includes: U.S. patent application Nos. 20030100023, 20030036092, 20030096226, and 20030091995, and the following publications: Feldhaus et al. Nat Biotechnol. 2003 21:163-70; Isticato et al. J Bacteriol. 2001 183:6294-301. Rode et al. Biotechniques. 1996 21:650, 652-3, 655-6, 658; Gram et al Proc Natl Acad Sci U S A. Apr. 15, 1992;89(8):3576-80; Marks et al. 1992 Bio/Technology. 10:779; Clackson et al. 1991. Nature. 352:624; Hawkins 1992 J. Mol. Biol. 226;889; Low et al 1996. J. Mol. Biol. 260,359; Barbas et al 1994 Proc. Natl. Acad. Sci. USA. 91:3809; Yang et al 1995 J. Mol. Biol. 254:392 and Balint et al 1993 Gene. 137:109.

### SUMMARY OF THE INVENTION

The invention provides methods for screening for a monoclonal antibody with a desirable activity. The methods involve altering a nucleic acid encoding a selected parental humanized monoclonal antibody to make a library of nucleic acids, introducing the library into mammalian cells such that a library of monoclonal antibodies are produced on the surfaces of the mammalian cells, and sorting the cells to isolate a cell producing a humanized monoclonal antibody with a desirable activity, e.g., increased affinity for an binding partner as compared to the parental antibody. This isolated cell may be cultured and used to reiterate the method. The subject methods find use in a variety of different industrial, medical and research applications.

### BRIEF DESCRIPTION OF THE DRAWINGS

FIG. 1 is a flow chart that schematically shows one embodiment of the invention.

FIG. 2 shows the sequences of the 5′ and 3′ ends of rabbit immunoglobulin heavy and light chains. The sequences set forth on FIG. 2, are SEQ ID NOS: 1-8, ordered from top to bottom.

FIG. 3A is a sequence alignment of three rabbit V_{H}1 allotypes (Rab_) with three human V_{H}1 sequences (Hu_). RAB_VH1A1: SEQ ID NO:9, RAB_VH1A2: SEQ ID NO:10, RAB_VH1A3: SEQ ID NO:11, Hu_VH_{—}3_{—}23: SEQ ID NO:12, Hu_VH_{—}3_{—}53: SEQ ID NO:12, Hu_VH_{—}3_{—}66: SEQ ID NO: 14, FIG. 3B is a sequence alignment of four rabbit Vk sequences with human Vk sequences. Rab_b4VK1: SEQ ID NO:15, Rab_b5VK1: SEQ ID NO:16, Rab_b9VK1: SEQ ID NO:17, Rab_basVk2: SEQ ID NO:18; HuVKI_o2: SEQ ID NO:19, HuVKI_L19: SEQ ID NO:20, HuKIL11: SEQ ID NO:21, VKIII_L16: SEQ ID NO:22.

FIG. 4 shows a schematic strategy for the CDR grafting of the rabbit B1 heavy chain immunoglobulin.

FIGS. 5A and 5B show sequence alignment showing resurfaced rabbit framework regions. RabBH1-a1: SEQ ID NO:23, V3-33: SEQ ID NO:24, RabBH1-a2: SEQ ID NO:25, 4-59/DP-71: SEQ ID NO:26, RabBH1-a3: SEQ ID NO:27, VL kappa: SEQ ID NO:28 L11: SEQ ID NO:29.

FIG. 6 is a schematic diagram-of mammalian cell display by expression of antibody genes as linear DNA.

FIG. 7 is a schematic diagram of mammalian cell display by expression of antibody genes in retroviral vector.

### DEFINITIONS

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988); Bird et al., Science, 242, 423-426 (1988); see Hood et al., “Immunology”, Benjamin, N.Y., 2nd ed. (1984), and Hunkapiller and Hood, Nature, 323, 15-16 (1986)).

An immunoglobulin light or heavy chain variable region consists of a “framework” region interrupted by three hypervariable regions, also called “complementarity determining regions” or CDRs. The extent of the framework region and CDRs have been precisely defined (see, “Sequences of Proteins of Immunological Interest,” E. Kabat et al., U.S. Department of Health and Human Services, (1983)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments-of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, unless otherwise indicated or clear from the context, antibody domains, regions and fragments are accorded standard definitions as are well known in the art. See, e.g., Abbas, A. K., et al., (1991) Cellular and Molecular Immunology, W. B. Saunders Company, Philadelphia, Pa.

As used herein, the term “humanized antibody” or “humanized immunoglobulin” refers to an antibody comprising one or more CDRs from an animal antibody, the antibody having been modified in such a way so as to be less immunogenic in a human than the parental animal antibody. An animal antibody can be humanized using a number of methodologies, including chimeric antibody production, CDR grafting (also called reshaping), and antibody resurfacing.

As used herein, the term “murinized antibody” or “murinized immunoglobulin” refers to an antibody comprising one or more CDRs from an animal antibody, the antibody having been modified in such a way so as to be less immunogenic in a mouse than the parental animal antibody. An animal antibody can be murinized using a number of methodologies, including chimeric antibody production, CDR grafting (also called reshaping), and antibody resurfacing.

The terms “affinity” and “avidity” have the same meaning and may be used interchangeably herein.

It is understood that the humanized antibodies designed and produced by the present method may have additional conservative amino acid substitutions which have substantially no effect on antigen binding or other antibody functions. By conservative substitutions is intended combinations such as gly, ala; val, ile, leu; asp, glu; asn, gln; ser, thr; lys, arg; and phe, tyr.

As used herein, the terms “determining,” “measuring,” and “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein the term “isolated,” when used in the context of an isolated antibody, refers to an antibody of interest that is at least 60% free, at least 75% free, at least 90% free, at least 95% free, at least 98% free, and even at least 99% free from other components with which the antibody is associated with prior to purification.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5α (amino) terminus and a translation stop codon at the 3α (carboxy) terminus. A coding sequence can include, but is not limited to, cDNA from viral, procaryotic or eucaryotic mRNA, genomic DNA sequences from viral or procaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., infra; DNA Cloning, supra; Nucleic Acid Hybridization, supra.

Two nucleic acid fragments are considered to “selectively hybridize” as described herein when they detectably pair with each other. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” a second polynucleotide if it has the same or substantially the same nucleotide sequence as a region of the second polynucleotide, its cDNA, complements thereof, or if it displays sequence identity as described above. A first polynucleotide may be derived from a second polynucleotide if the first polynucleotide is used as a template for, e.g. amplification of the second polynucleotide.

A first polypeptide is “derived from” a second polypeptide if it is (i) encoded by a first polynucleotide derived from a second polynucleotide, or (ii) displays sequence identity to the second polypeptides as described above. The term “unit dosage form” as used herein refers to physically discrete units suitable as unitary dosages for subjects (e.g., animals, usually humans), each unit containing a predetermined quantity of an agent, e.g. an antibody in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

A polynucleotide is “derived from” a particular cell if the polynucleotide was obtained from the cell. A polynucleotide may also be “derived from” a particular cell if the polynucleotide was obtained from the progeny of the cell, as long as the polynucleotide was present in the original cell. As such, a single cell may be isolated and cultured, e.g. in vitro, to form a cell culture. A nucleotide isolated from the cell culture is “derived from” the single cell, as long as the nucleic acid was present in the isolated single cell.

The terms “treatment” “treating” and the like are used herein to refer to any treatment of any disease or condition in a mammal, e.g. particularly a human or a mouse, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a variable domain” includes reference to one or more variable domains and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF PREFERRED EMBODIMENTS

The invention provides methods for screening for a monoclonal antibody with a desirable activity. The methods involve altering a nucleic acid encoding a selected parental humanized monoclonal antibody to make a library of nucleic acids, introducing the library into mammalian cells such that a library of monoclonal antibodies are produced on the surfaces of the mammalian cells, and sorting the cells to isolate a cell producing a humanized monoclonal antibody with a desirable activity, e.g., increased affinity for an binding partner as compared to the parental antibody. This isolated cell may be cultured and used to reiterate the method. The subject methods find use in a variety of different industrial, medical and research applications.

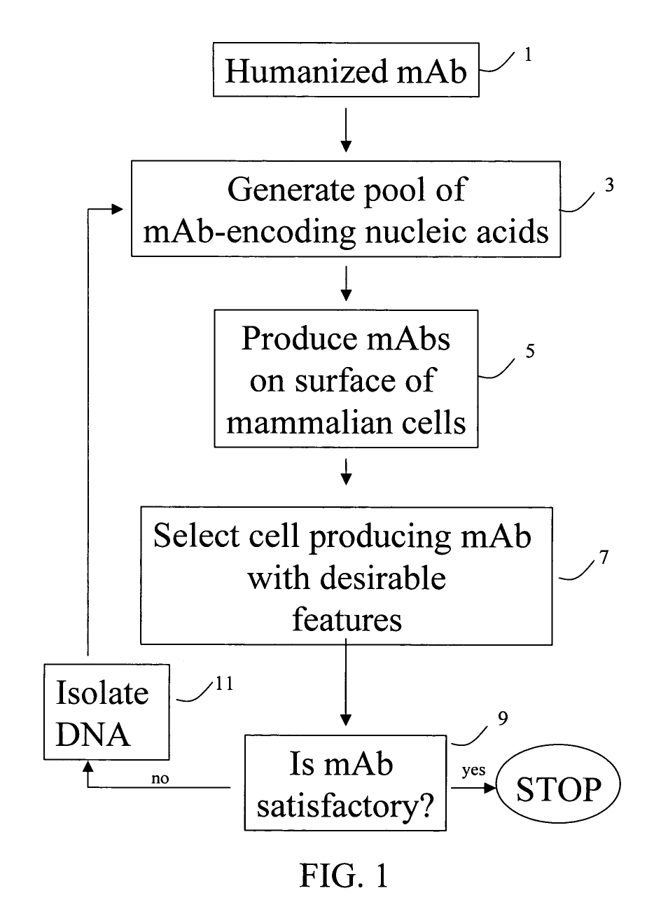

Certain embodiments of the methods described herein are set fort in the flow diagram of FIG. 1. In general, the methods involve selecting a parental monoclonal antibody with a desirable property 1 (e.g., affinity for an antigen), mutating the nucleic acids for that monoclonal antibody to generate a library of monoclonal antibody (mAb)-encoding nucleic acids 3, introducing the pool of nucleic acids into mammalian cells using a linear DNA or retroviral expression system to produce the modified mAbs on the surfaces of mammalian cells 5, and isolating cells that produce a monoclonal antibody with an improved property (e.g. a greater affinity for the same antigen) 7. If that antibody is not satisfactory for future use 9, then the antibody encoding nucleic acids may be isolated from the isolated cells, transferred into cells, and the cells subjected to a further round of selection 11. Optionally, nucleic acids that encode a monoclonal antibody produced by an isolated cells may be mutated and used as a starting point to reiterate the process.

In further describing the subject invention, the methods of the invention will be described first, followed by a review of the representative applications in which the subject methods find use.

Methods for Producing a Library of Monoclonal Antibodies

The invention provides methods for producing a library of monoclonal antibodies. In general, the methods involve altering a nucleic acid encoding a selected monoclonal antibody (a “parental” monoclonal antibody) to produce a library of variant nucleic acids that encode different variants of the parental monoclonal antibody (the variants are also called “modified” or “daughter” monoclonal antibodies), and introducing the library of variant nucleic acids into mammalian cells to produce a library of monoclonal antibodies that are variants of the parental monoclonal antibody. In most embodiments, the variant monoclonal antibodies are produced in mammalian cells and are anchored to the surface of the cells, and available for binding of an antigen that is outside of the cell.

Parental Monoclonal Antibodies

As mentioned above, the starting material of the described process is nucleic acid encoding a monoclonal antibody that has been selected because of a desirable activity. Such desirable activities include binding activities, such as a high affinity binding to an antigen, specific binding to an antigen, a catalytic activity, a blocking activity, or a particular therapeutic activity. Methods for making and selecting monoclonal antibodies, including rabbit monoclonal antibodies are generally well known in the art (see, e.g., Harlow et al., Antibodies: A Laboratory Manual, First Edition (1988) Cold spring Harbor, N.Y.; Spieker-Polet et al. 1995 Proc Natl Acad Sci 92: 9348-52), and need not be described here in any detail.

In many embodiments, the starting material of the described process is a humanized, or, in some embodiments, a murinized monoclonal antibody. In other words, a suitable selected monoclonal antibody may comprise one or more CDRs from an animal antibody, the antibody having been modified in such a way so as to be less immunogenic in a human or mouse than the parental animal antibody. As is known, animal antibodies can be humanized using a number of methodologies, including chimeric antibody production, CDR grafting (including reshaping), and antibody resurfacing. In general, chimeric antibodies are made by transferring the constant regions from a human antibody onto an antibody from a non-human animal, and CDR grafting involves transferring CDR regions, corresponding to the domains that provide specific binding, from a non-human antibody onto a human antibody framework. Resurfacing involves substituting framework amino acids that are exposed in a non-human antibody (i.e., on the exterior surface of the antibody) with equivalent exposed residues of a human antibody. Suitable methods for humanizing or murinizing antibodies may be found in U.S. Pat. Nos. 6,331,415 B1, 5,225,539, 6,342,587, 4,816,567, 5,639,641, 6,180,370, 5,693,762, 4,816,397, 5,693,761, 5,530,101, 5,585,089, 6,329,551, and, in particular in U.S. Patent Application No. 60/404,117, filed Aug. 15, 2002, which is specifically incorporated herein by reference in its entirety. In most embodiments, rabbit monoclonal antibodies are used in the subject methods.

The subject parental antibodies are not usually associated with viral sequences, and usually have immunoglobulin heavy and light chains that have been “naturally paired” by the immune system of the host. As such, in most embodiments, the subject parent monoclonal antibodies essentially maintain the combination of heavy and light chains that are present in the antibody producing cell from which monoclonal antibody is derived.

Nucleic acids encoding a subject monoclonal antibody are usually obtained from a cell, usually a hybridoma cell, that produces the monoclonal antibody. Such nucleic acids may be isolated using routine methods (e.g., Ausubel, et al, Short Protocols in Molecular Biology, 3rd ed., Wiley & Sons, 1995; Sambrook, et al, Molecular Cloning: A Laboratory Manual, Third Edition, 2001 Cold Spring Harbor, N.Y.).

For example, sequences encoding heavy and light chains may be amplified from the cDNA using techniques well known in the art, such as Polymerase Chain Reaction (PCR). See Mullis, U.S. Pat. No. 4,683,195; Mullis et al., U.S. Pat. No. 4,683,195; Polymerase Chain Reaction: Current Communication in Molecular Biology, Cold Springs Harbor Press, Cold Spring Harbor, N.Y., 1989. Briefly, cDNA segments encoding the variable domain of the antibody are exponentially amplified by performing sequential reactions with a DNA polymerase. The reaction is primed by a 5′ and a 3′ DNA primer. In some embodiments, the 3′ antisense primer corresponding to a DNA sequence in the constant (or joining) region of the immunoglobulin chain and the 5′ primer (or panel of related primers) corresponding to a DNA sequence in the variable region of the immunoglobulin chain. This combination of oligonucleotide primers has been used in the PCR amplification of murine immunoglobulin cDNAs of unknown sequence (see Sastry et at., Proc Natl. Acad. Sci. 86:5728-5732, 1989 and Orlandi et al., Proc. Natl. Acad. Sci. 86:3833-3837, 1989). Alternatively, an “anchored polymerase chain reaction” may be performed (see Loh et al., Science 243:217-220, 1989). In this procedure, the first strand cDNA is primed with a 3′ DNA primer as above, and a poly(dG tail) is then added to the 3′ end of the strand with terminal deoxynucleotidyl transferase. The product is then amplified by PCR using the specific 3′ DNA primer and another oligonucleotide consisting of a poly(dC) tail attached to a sequence with convenient restriction sites. In many embodiments, however, the entire polynucleotide encoding a heavy or light chain is amplified using primers spanning the start codons and stop codons of both of the immunoglobulin cDNAs, however, depending on the amplification products desired, suitable primers may be used. Typical primers for use with rabbit monoclonal antibodies are as follows: heavy chain, 5′ end (CACCATGGAGACTGGGCTGCGCTGGCTTCTCCTGGTCGCTGTG; SEQ ID NO:30); heavy chain, 3′ end (CTCCCGCTCTCCGGGTAAATGAGCGCTGTGCCGGCGA;-SEQ ID NO:31); light chain kappa, 5′end (CAGGCAGGACCCAGCATGGACACGAGGGCCCCCACT; SEQ ID NO:32); and L kappa, 3′end (TCAATAGGGGTGACTGTTAGAGCGAGACGCCTGC; SEQ ID NO:33). Suitable restriction sites and other tails may be engineered into the amplification oligonucleotides to facilitate cloning and further processing of the amplification products. Amplification procedures using nested primers may also be used, where such nested primers are well known to one of skill in the art.

Mutagenesis

In most embodiments of the subject methods, the nucleic acids encoding the subject parental monoclonal antibody are altered by mutagenesis to provide a library of candidate variant monoclonal antibody-encoding nucleic acids. A library may encode at least 2 (i.e., at least about 5, at least about 10, at least about 50, at least about 100, at least about 200, at least about 500, at least about 1000, at least about 5000, at least about 10,000 or at least about 50,000 or more, usually up to about 100,000 or more) variants of the parental monoclonal antibody.

Mutagenesis may be random mutagenesis, or may make directed changes in the antibody-encoding nucleic acids. In most embodiments, depending on the desired changes, a nucleic acid encoding a region of an antibody, e.g., one or more CDR regions, one or more a framework regions, a heavy or light chain variable domain, or any subdomain, any individual amino acid or combination thereof, in a heavy and/or light chain immunoglobulin-encoding nucleic acid may be altered. In most embodiments, the parental nucleic acid is altered to produce a plurality of variant nucleic acids containing at least one (e.g. two, three, four, or five or more) changes such that an encoded antibody region is changed by at least one (e.g. two, three, four, or five or more) amino acids in comparison to the equivalent region in the parental antibody.

In particular embodiments, nucleic acids encoding at least one of the CDR and framework regions of a parental antibody are subjected to random mutagenesis to produce a library of variant antibodies containing, collectively, substitutions, additions or deletions of the amino acids that span the region. In other embodiments, nucleic acids encoding particular amino acids in a framework region or CDR of a parental antibody is subjected to random mutagenesis to produce a library of antibody variants that collectively has substitutions, deletions or additions of particular amino acids. Also, in specific embodiments, nucleic acid encoding particular amino acids in a framework or CDR region is directionally altered to provide a library of antibody variants that are collectively altered at the particular amino acids such that the amino acids are substituted for a specific nucleic acid. In other words, a subject library may contain antibody-encoding nucleic acids that are altered at any position across a antibody region, or in individual position thereof.

In particular embodiments, particularly those that involve parental antibodies that are non-human antibodies that have been humanized by resurfacing or CDR grafting, the individual amino acids that were substituted during humanization may be individually back-altered to become identical to amino acids at equivalent positions in the parental non-human antibody. In other words, if a resurfaced antibody contains 20 amino acid changes in comparison to its parental non-resurfaced counterpart, each of the 20 changes could be back-mutated to the amino acid that was present at the equivalent position in the parental non-resurfaced antibody. A library containing such variants usually contains antibodies that, collectively, contain all changes at all of the desired positions, and each antibody usually contains an alteration in at least 1, at least 2, at least 3, at least 4, at least 5, at least about 7, at least about 10, at least about 15, or at least about 20 or more positions.

To generate a library of variant monoclonal antibodies from a parental monoclonal antibody, the coding sequence of parental monoclonal antibody may be mutated so as to provide the library of variant monoclonal antibodies. The immunoglubulin heavy or light chain coding sequences may be mutated in various ways known in the art to generate targeted or random changes in the sequence of the encoded antibody. The sequence changes may be substitutions, insertions, deletions, or one or more (e.g., 2, 3, 4, 5, 6 or 7 or more residues), or a combination thereof.

Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:35-41; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4.

In many embodiments, it is desirable to screen a large number of candidate monoclonal antibodies in order to rapidly identify an altered monoclonal antibody with an improved activity over the parental antibody. Any convenient protocol for generating large numbers of mutant proteins from an initial wild type protein may be employed, where representative protocols are well known to those of skill in the art and of interest include, but are not limited to: (1) error prone PCR, as described in U.S. Pat. No. 6,358,709 (the disclosure of which are herein incorporated by reference); (2) DNA shuffling, as described in U.S. Pat. Nos. 6,355,484; 6,365,408 (the disclosure of which are herein incorporated by reference); (3) in vivo mutagenesis in mutator cells (e.g., mutator bacterial strains deficient in mismatch repair enzymes) or using mutational vectors, as described in U.S. Pat. Nos. 6,004,804; 6,146,894; 6,165,718; 6,211,351; (the disclosures of which are herein incorporated by reference); (4) directed evolution protocols, as described in U.S. Pat. No. 6,358,709, 5,723,323 and 6,171,820 (the disclosures of which are herein incorporated by reference); (4) directed mutation, as described in U.S. Pat. Nos. 5,702,931; 5,932,419; 5,935,830; the disclosures of which are herein incorporated by reference; and the like. Oligonucleotides of varying sequence may also be inserted into an antibody-coding sequence to provide such a library of candidate monoclonal antibodies. Such protocols result in the production of a population of candidate monoclonal antibodies. The generated population of candidate monoclonal antibodies is then screened, using the methods described in the next section, in order to identify a monoclonal antibody that has an improved activity over the parental antibody used to generate the library.

Expression

In general, the nucleic acids encoding the variant polypeptides are incorporated into an expression cassette for their expression in a mammalian host cell. In most embodiments, each expression cassette is more than about 0.5 kb in length, more than about 1.0 kb in length, more than about 1.5 kb in length, more than about 2 kb in length, more than about 4 kb in length, more than about 5 kb in length, and is usually less than 10 kb in length. An expression cassette may be linear, or encompassed in a circular vector, usually a viral vector such as a retroviral vector.

Each expression cassette will typically further include expression control DNA sequences operably linked to the immunoglobulin coding sequences to form heavy and light chain expression cassettes. In some embodiments, the expression control sequences will be eukaryotic promoter capable of directing expression of the immunoglobulin heavy or light chain polypeptide in eukaryotic host cells. In certain embodiments, a human cytomegalovirus (HCMV) promoter and/or enhancer and/or terminator is used to direct expression of the polypeptides in mammalian cells. Suitable promoters, terminators, and translational enhancers suitable for expression of immunoglobulin heavy and light chains are known in the art, and many are discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). Suitable promoters include SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In many embodiments of the invention, the heavy and light chain expression cassettes are linear expression cassettes, or are present on a circular nucleic acid (e.g. a circular vector, for example a retroviral vector). Linear expression cassettes are typically not part of a circular vector and are not otherwise associated with vector sequences such as an origin of replication, or vector backbone. In certain embodiments, however, the linear expression cassette may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosporibosyl transferase gene, CAD, the adenosine deaminase gene, the asparagine synthetase gene, the antibiotic resistance genes, e.g. tet^{r}, amp^{r}, Cm^{r} or cat, kan^{r} or neo^{r} (aminoglycoside phosphotransferase genes), the hygromycin B phosphotransferase gene, and the like.

In most embodiments, the linear expression cassette is a non-integrative polynucleotide, i.e., it does not integrate into a genome of a host cell, and, as such, typically does not contain recombination sites or flanking sequences to facilitate homologous recombination.

In certain embodiments, the heavy and light chain coding sequences are present on the same plasmid, e.g., an autonomously replicating or viral expression vector, e.g. a retroviral expression vector, and expression of the two chains may be accomplished by using a single promoter and an internal ribosome entry site (IRES) between the two coding sequences. Such constructs are known to those of skill in the art, see, e.g., Dirks et al (Gene; 128:247-9, 1993).

In many embodiments, the expression cassettes further comprise a cell surface targeting region which, when operably linked to an immunoglobulin heavy or light chain polypeptide, directs the polypeptide to the surface of a mammalian cell. In other words, an expression cassette provides for targeting of the antigen to the surface of the host cell by producing an immunoglobulin operably linked to a cell surface targeting polypeptide. In such embodiments, an immunoglobulin heavy or light encoding nucleic acid may be operably linked to a cell surface targeting polypeptide-encoding nucleic acid in the expression cassette, and transcription and subsequent translation of the nucleic acids provides for production of a fusion protein containing an immunoglobulin chain and the cell surface targeting polypeptide. As such, the expression cassette can provide for targeting of an antigen to the surface of a mammalian host cell. Suitable cell surface targeting polypeptides and their encoding nucleic acid sequences may be those of, for example, transmembrane serine threonine or tyrosine kinase receptors or receptor transmembrane domains, such as the epidermal growth factor receptor (EGFR) transmembrane domain (Ullrich, A. et al. Nature 309: 418425 (1984)). Further examples of strategies for targeting of polypeptides in a cell or protein secretion may be found in U.S. Pat. No. 6,455,247. In most embodiments, the cell surface-targeting polypeptide is operably linked to proximal to or at the C-terminus of a variant antibody.

Membrane-anchoring sequences are well known in the art and are based on the genetic geometry of mammalian transmembrane molecules. Peptides are inserted into the membrane based on a signal sequence and usually require a hydrophobic transmembrane domain. The transmembrane proteins are usually inserted into the membrane such that the regions encoded N-terminal of the transmembrane domain are extracellular and the sequences C-terminal become intracellular. Such membrane anchoring sequences are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane-anchoring sequences are known for a wide variety of proteins and any of these may be used. Particularly preferred membrane-anchoring sequences include, but are not limited to, those derived from CD8, ICAM-2, IL-8R, CD4, LFA-1 and the PDGF receptor transmembrane regions. Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class 11 integral membrane-proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP; (SEQ ID NO:34) Nakauchi et al., PNAS USA 82:5126 (1985) and 1-21 in the case of ICAM-2 (MSSFGYRTLTVALFTLICCPG; (SEQ ID NO:35) Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR; (SEQ ID NO:36) Nakauchi, supra) and 224-256 from ICAM-2 (MVIIVTVVSVLLSLFVTSVLLCFIFGQHLRQQR; (SEQ ID NO:37) Staunton, supra). Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT, (SEQ ID NO:38) with the bolded serine the site of the anchor, see Homans et al., Nature 333(6170):269-72 (1988), and Moran et al., J. Biol. Chem. 266:1250 (1991)). In order to do this, the GPI sequence from Thy-1 can be placed in a position at the 3′ end of the antibody-encoding nucleic acid.

In certain embodiments, a flexible linker may separate an immunoglobulin domain from an operably linked cell-surface targeting domain. Useful linkers include glycine-serine polymers (including, for example, (GS)ₙ, (GSGGS)ₙ and (GGGS)ₙ, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In alternative embodiments, an antibody may be presented on the surface of a mammalian cell in which it is produced using a non-covalent specific interaction between a portion of the antibody (e.g. an non-antigen binding portion of an antibody) and a binding partner molecule that is present on the cell surface. For example, a divalent antibody may be a binding partner that is bound to the surface of a mammalian cell by one of its Fab regions binding a molecule on the surface of the second parental cell. The other Fab region of the divalent antibody may bind to the antibody that is produced by the mammalian cell to tether it to the surface of the mammalian cell. Another example of a binding partner is an antibody that is modified, e.g., with biotin, such that it specifically binds a parental cell, e.g., one that expresses streptavidin on its surface. In this example, a non-antigen binding portion of the antibody produced by, e.g. secreted by, a mammalian cell will bind to the biotinylated, surface bound antibody, to anchor the antibody produced by the mammalian cell to the mammalian cell. As such, a first antibody may also be used to anchor a second antibody to the surface of a cell in which the second antibody is expressed. In certain embodiments, therefore, a biotinylated goat or mouse anti-rabbit antibody may be used to anchor a rabbit antibody produced (e.g., secreted) by a cell to the exterior surface of the cell by producing streptavidin on the exterior surface of the cell. Streptavidin may be targeted to the exterior surface of a cell using the transmembrane domains described above. As an alternative, cells can be biotinylated, and straptavidin can be bound to cell surface biotins. To prevent secreted antibodies from diffusing to neighboring cells, gel-like reagents such as agarose or gelatin can be employed to restrict molecular diffusion.

Suitable cells for expression of the surface targeted variant monoclonal antibodies include cells from mammals, e.g., mouse, rabbit, hamster, human etc. or avians, e.g., chicken, since animal cells usually ensure correct post-transcriptional modification, correct protein targeting, and correct protein conformation. In certain embodiments, the host cell is a human cell (e.g., HeLa), a mouse cell (e.g., NIH 3T3), a chicken cell (e.g., DT-40) or a rabbit cell (e.g., 240E). Other exemplary mammalian cells include monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209. In other embodiments, the suitable cells for antibody expression are cells for packaging virus, especially retroviral packaging cells, such as EcoPack2-293, Amphopack-293, Retropack PT67, GP2-293 cells, as sold by Clontech (Palo Alto, Calif.). Other examples include ecotropic retroviral packaging cell line, GP+E86, and the amphotropic packaging cell line, GP+EnvAm12 (AM-12, GenPak™) from Genetix Pharmaceuticals and Viraport packaging cell lines from Stratagene.

Expression cassettes present in linear DNAs or viral, e.g., retroviral vectors may be introduced into a host cell using a variety of methods, including viral infection, transfection, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e., in vitro). A general discussion of these methods can be found in Ausubel, et al, Short Protocols in Molecular Biology, 3rd ed., Wiley & Sons, 1995.

In order to assemble a linear or viral vectors, i.e. to operably link the coding sequences with any other coding or regulatory sequences, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) may be used. Several methods are known in the art for producing antibody-encoding nucleic acids, including those found in U.S. Pat. Nos. 6,180,370, 5,693,762, 4,816,397, 5,693,761 and 5,530,101. One PCR method utilizes “overlapping extension PCR” (Hayashi et al., Biotechniques. 1994. 312, 314-5) to create expression cassettes for the heavy and light chain encoding nucleic acids. In this method multiple overlapping PCR reactions using the cDNA product obtained from the antibody producing cell and other appropriate nucleic acids as templates generates an expression cassette.

Depending on the constant regions and other regions used, several types of antibody that are known in the art may be made by this method. As well as full length antibodies, antigen-binding fragments of antibodies may be made. These fragments include, but are not limited to, Fab, Fab′ and F(ab′)₂, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv minibodies, Fab minibodies, and dimeric scFv and any other-fragments comprising a V_{L} and a V_{H} domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH₁, CH₂, CH₃, transmembrane, and/or cytoplasmic domain, on the heavy chain, and a light chain constant domain, e.g., a Ckappa or Clambda domain, or portion thereof on the light chain. Also included in the invention are any combinations of variable region(s) and CH₁, CH₂, CH₃, Cₖₐₚₚₐ, C_{lambda}, transmembrane and cytoplasmic domains.

Production of circular vectors for expression of antibodies is well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Certain recombination-based methods, e.g. GATEWAY™ (InVitrogen, Carlsbad, Calif.)), CREATOR™ (Clontech, Palo Alto, Calif.) or ET cloning (Muyrers et al, Nucleic Acids Res. 27:1555-7 (1999)) methodologies may also be used in the production of the subject expression cassettes.

After expression cassettes corresponding to the heavy and light chain nucleic acids isolated from a single antibody producing cell, or a cell population, has been introduced into cells (e.g., a pair of linear molecules, each containing an expression cassette for the heavy or light chain, or a plasmid or viral vector containing expression cassettes for both the heavy and light chains), the cells are typically incubated, normally at 37° C., sometimes under selection, for a period of about 1-48 hours in order to allow for the expression of the antibody. In most embodiments, the antibody is produced such that it is present on the surface of a cell in which it is produced.

As such, by creating a population of antibody heavy and light chain-encoding nucleic acids that encode variants of a parental monoclonal antibody and providing for the expression of the encoded antibodies on the surfaces of mammalian cells, a library of variant monoclonal antibodies may be produced. In some embodiments, e.g. when the copy number of the expression cassettes are controlled, such as in the case of retroviral vector, or, to a certain extent linear vectors and plasmid vectors, a cell producing an antibody will produce a single member of the library of variant antibodies. However, depending on how many heavy and light chain-encoding nucleic acids are introduced into the cell, a cell may produce more than one, e.g., 2, 3, 4, or 5 or more the variant antibodies. In this situation, molecular shuffling, i.e., pairing of variant heavy chain and variant light chain molecules, between different variants of heavy and light chains will happen. This is a unique advantage that most other display systems (such as phage display) do not have. In other words, a cell used in the instant methods may produce more than one variant of the parental antibody, depending on how many different nucleic acids are introduced into the cell.

In certain embodiments, the variant heavy chain encoding nucleic acids and the variant light chain encoding nucleic acids may be transferred into mammalian cells such that both the light chain and the heavy chains produced have a different sequence to the heavy and light chains of the parental antibody.

Compositions

The subject invention also provides compositions, such as a library of mammalian cells displaying a plurality of different monoclonal antibodies (e.g., 2 or more, 3 or more, 4 or more, 5 or more, 10 or more, 20 or more or 50 or more, etc.), a library of mammalian cells displaying monoclonal antibodies, where each cell displays a different monoclonal antibody on its surface, a library of expression cassettes, for example linear or circular (e.g., in a retroviral vector) expression cassettes that provide for expression of a library of monoclonal antibodies in mammalian cells.

Methods for Identifying a Monoclonal Antibody of Interest

The invention also provides methods of identifying a monoclonal antibody of interest. In general, the methods involve producing a library of candidate monoclonal antibodies using the methods described above, and screening the library to identify a monoclonal antibody of interest. In many embodiments, screening involves identifying an antibody in the library that has an improved activity as compared to the parental antibody. For example, a monoclonal antibody of interest may exhibit: stronger binding to an binding partner (e.g., an increased affinity to a binding partner such as a protein or cell as compared to the parental antibody), greater specificity (e.g., binds to a subset of the binding partners of the parental antibody), stronger expression (e.g., is expressed at higher levels, e.g., greater than about 20%, 50%, 100%, 500% or 1000% or more, in a mammalian cell) or any other activity (i.e., inhibition of binding between two binding partners), as compared to a parental monoclonal antibody.

In embodiments where a monoclonal antibody of interest has greater affinity for a binding partner than a parent monoclonal antibody, affinity for the binding partner is increased by at least about 20%, at least about 50%, at least about 100%, at least about 5-fold, at least about 10-fold, at least about 20-fold, at least about 50-fold or at least about 100-fold or more. For example, if a parental monoclonal antibody has an affinity for a binding partner of about 1×10⁻⁸ M, a monoclonal antibody of interest derived from that parental monoclonal antibody will have a binding affinity of about 0.5×10⁻⁸ M, about 1×10⁻⁹ M, about 0.5×10⁻⁹ M, or about 1×10⁻¹⁰ M or more.

Since the antibodies are produced on the surfaces of mammalian cells, an antibody of interest is generally identified by isolating a cell producing the antibody using any one or more of a variety of positive or negative cell selection methods. In general, therefore, a library of cells producing candidate monoclonal antibodies is made using the methods described above, and cells are isolated based on an activity of the antibody which it produces.

Positive and negative selection assays include those in which a binding partner, e.g., an antigen such as a polypeptide to which the parental antibody binds, is mixed with the cell population under conditions sufficient for binding of the binding partner to the antibodies produced by the cells, and the cells that bind to the binding partner are isolated from the rest of the cell population, either because they bind or do not bind to the binding partner. This selection may be done by a variety of means, and generally involves a binding partner that is either detectably labeled, or bound to a solid support. In other words, a cell producing a monoclonal antibody of interest may be selected based on its binding to a binding partner, or, in other embodiments, a monoclonal antibody of interest may be selected based on its lack of binding to a binding partner.

In some embodiments, cells producing a monoclonal antibody of interest may be isolated from the rest of the population of cells by their binding to a solid support. For example, cells producing a monoclonal antibody of interest may be separated from the population of cells using magnetic separation using paramagnetic particles that are coated with a binding partner (Kiesel et al. euk Res. 1987 11:1119-25). Paramagnetic particles are available with a variety of surface derivatization chemistries to allow for the covalent attachment of a wide range of binding partners, e.g. a protein of interest or a cell producing the same on its surface. Mammalian cells producing surface-bound antibodies with high affinity for the binding partner bind to the magnetic particles and may be isolated in a strong magnetic field that attracts the magnetic beads. Alternatively, cells that bind to the particles may be separated in a continuous magnetic separator. In other embodiments, the variant antibody cell population is contacted with a solid support in which a binding partner is chemically immobilized, and cells that display antibodies that bind to the binding partner are retained on the solid support. Following washing, cells which specifically bound to the support may be released using binding partner that is not immobilized to the support.

In alternative embodiments, cells producing a monoclonal antibody of interest may be separated from the population of cells by cell sorting. In these embodiments, fluorescence activated cell sorting (FACS) may be used. FACS permits the separation of subpopulations of cells on the basis of their light scatter properties as they pass through a laser beam. Since the cells producing a monoclonal antibody of interest may be bound to a florescent-tagged binding partner, they are identified by FACS as being fluorescent and can be separated from the non-fluorescent cells. In certain embodiments, a cell producing an antibody of interest binds to more fluorescent binding partner than other cells in the population and, as such, may be identified because it exhibits greater fluorescence. In other words, a cell producing an antibody with a higher affinity for an antigen, an antibody that is more highly expressed on the cell, an antibody that is more specific for an antigen, etc., will be identified in a cell population because it exhibits more fluorescence than the other cells.

As such, in certain embodiments, a FACS cell sorter may be calibrated using a population of cells producing a parental antibody to identify a range of normal values for cells, and cells from the population of cells producing the variant antibodies may be selected if they exhibit fluorescence outside of this range. Alternatively, a population of cells producing variant antibodies may be analyzed using FACS, and the cells exhibiting the most fluorescence in the population may be separated from the rest of the cells in the population. For example, about 1%, about 5%, about 10%, about 15% or about 20% of the cells may be selected because they exhibit more fluorescence than the rest of the cells.

Depending on the exact methodology used, labeled antigen may be added to the cell population at a non-saturating amount, or in combination (at a certain ratio) with unlabeled antigen, to facilitate the identification cells producing antibodies of interest. In other embodiments, a distinguishably labeled second binding partner which binds to all the monoclonal antibodies produced by the cells with equal affinity may be added to the cell population to identify the expression level of an antibody in a single cell. Using the distinguishably labeled second binding partner, e.g., a labeled antibody that binds to an Fc region of the monoclonal antibody variants, the amount of binding of the first binding partner can be normalized to the amount of a monoclonal antibody produced by a cell. In other embodiments, inhibitors non-specific protein binding, e.g., BSA or dried milk solution, may be added to the cell population to facilitate identification of cells producing antibodies with desired properties.

Cells producing a monoclonal antibodies of interest, once isolated from the population of cells may be cultured and subjected to further rounds of selection or used to isolate the nucleic acids encoding the monoclonal antibodies of interest, by e.g., plasmid rescue (if a plasmid vector is used), PCR (if a linear vector is used), or by isolation of virus particles produced by the cell (if a viral vector is used). These nucleic acids may be used in a variety of ways. For example, the nucleic acids may be used to produce a monoclonal antibody of interest, as will be described below. In other embodiments, the nucleic acids may be used as starting materials for reiterating the above mutagenesis/selection process. In other words, the first monoclonal antibody of interest identified using the above methods may become a parental monoclonal antibody and the above methods may be repeated to produce a second monoclonal antibody of interest. In another embodiment, the nucleic acids isolated from the isolated cells may be directly transferred into a population mammalian cells so that the antibodies represented by the isolated nucleic acids are produced on the surfaces of the mammalian cells. This cell population may be mixed with antigen, and the cells that bind to antigen may be again isolated from other cells in the population, reiterating the selection process. This process may be re-iterated once, twice, thrice or 4, 5, 6, 7, 8 or more times until a monoclonal antibody of interest suitable for future is identified in the isolated cell population. The nucleic acids encoding a suitable monoclonal antibody of interest may be isolated and used to produce the monoclonal antibody of interest.

If a single retroviral vector with an IRES is used, the surface bound antibodies are usually produced using a first packaging cell, and cells producing a monoclonal antibody are isolated using the methods described above. Once the cells are isolated, the supernatant of the isolated cells may be used to infect a second packaging cell, and cells producing a monoclonal antibody are isolated using the methods described above. In other words, if a single retroviral vector with an IRES is used, the packaging cell lines used must be alternated in successive rounds of selection in order to perform the methods.

Methods of Producing a Monoclonal Antibody of Interest

The invention provides several methods of producing a monoclonal antibody of interest. In general these methods involve incubating a host cell containing a nucleic acid encoding a monoclonal antibody of interest under conditions sufficient for production of the antibody. In many embodiments, the methods of producing a monoclonal antibody of interest involve transferring identified expression cassettes for an monoclonal antibody of interest into a suitable vector, and transferring the recombinant vector into a host cell to provide for expression of the monoclonal antibody. In some embodiments, the subject methods involve transferring at least the variable domain-encoding sequences from the identified heavy and light chains into vectors suitable for their expression in immunoglobulin heavy and light chains. Suitable constant domain-encoding sequences and/or other antibody domain-encoding sequences may be added to the variable domain-encoding sequences at this point. These nucleic acid modifications may also allow for humanization of the subject antibody.

The subject monoclonal antibodies can be produced by any method known in the art for the synthesis of antibodies, in particular, by recombinant expression techniques.

Recombinant expression of a subject monoclonal antibody, or fragment, derivative or analog thereof, usually requires construction of an expression vector containing a polynucleotide that encodes the antibody. Methods which are well known to those skilled in the art can be used to construct expression vectors containing antibody coding sequences and appropriate transcriptional and translational control signals. These methods include, for example, in vitro recombinant DNA techniques and synthetic techniques. As such, the invention provides vectors comprising a nucleotide sequence encoding an antibody molecule of the invention.

The expression vector is transferred to a host cell by conventional techniques and the transfected cells are then cultured to produce a subject antibody. In most embodiments, vectors encoding both the heavy and light chains are co-expressed in the host cell to provide for expression of the entire immunoglobulin molecule.

A variety of host-expression vector systems may be utilized to express a subject monoclonal antibody. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells etc.) harboring recombinant expression constructs containing promoters derived from the genome of mammalian cells (e.g., metallothionein promoter) or from mammalian viruses (e.g., the adenovirus late promoter; the vaccinia virus 7.5K promoter). In many embodiments, bacterial cells such as Escherichia coli, and eukaryotic cells are used for the expression of entire recombinant antibody molecules. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express antibodies. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

Once produced, a monoclonal antibody of interest may be tested using a variety of assays, described below.

Binding Assays

In these assays, each antibody of a plurality of antibodies is tested for its ability to bind specifically to a substrate. The term “specifically” in the context of antibody binding, refers to high avidity and/or high affinity binding of an antibody to a specific antigen i.e., a polypeptide, or epitope. In many embodiments, the specific antigen is an antigen (or a fragment or subfraction of an antigen) used to immunize the animal host from which the antibody-producing cells were isolated. Antibody specifically binding an antigen or fragment thereof is stronger than binding of the same antibody to other antigens. Antibodies which bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity of 10⁻⁷ M or more, e.g., 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

Typically, in performing a screening assay, antibody samples produced by a library of antibody producing host cells are deposited onto a solid support in a way that each antibody can be identified, e.g. with a plate number and position on the plate, or another identifier that will allow the identification of the host cell culture that produced the antibody.

The antibodies of the invention may be screened for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally involve lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally involves preparation of protein samples followed by electrophoresis of the protein samples in a polyacrylamide gel (e.g., 8%-20% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-Tween 20), and incubated with primary antibody (the antibody of interest) diluted in blocking buffer. After this incubation, the membrane is washed in washing buffer, incubated with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I), and after a further wash, the presence of the antigen may be detected. One of skill in the art would be knowledgeable as to the parameters that can be modified to increase the signal detected and to reduce the background noise.

ELISAs involve preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Antibodies of the invention may be screened using immunocytochemisty methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific.

In certain embodiments, however, the assay is an antigen capture assay, and an array or microarray of antibodies may be employed for this purpose. Methods for making and using microarrays of polypeptides are known in the art (see e.g: U.S. Pat. Nos. 6,372,483, 6,352,842, 6,346,416 and 6,242,266).

Inhibitor Assays

In certain embodiments, the assay measures the specific inhibition of an antibody to an interaction between a first compound and a second compound (e.g. two biopolymeric compounds) or specifically inhibits a reaction (e.g. an enzymatic reaction). In the interaction inhibition assay, one interaction substrate, usually a biopolymeric compound such as a protein e.g. a receptor, may be bound to a solid support in a reaction vessel. Antibody is added to the reaction vessel followed by a detectable binding partner for the substrate, usually a biopolymeric compound such as a protein e.g. a radiolabeled ligand for the receptor. After washing the vessel, interaction inhibition may be measured by determining the amount of detectable binding partner present in the vessel. Interaction inhibition occurs when binding of the binding partner is reduced greater than about 20%, greater than about 50%, greater than about 70%, greater than about 80%, or greater than about 90% or 95% or more, as compared to a control assay that does not contain antibody.

In the reaction inhibition assay, an enzyme may be bound to a solid support in a reaction vessel. Antibody is usually added to the reaction vessel followed by a substrate for the enzyme. In many embodiments, the products of the reaction between the enzyme and the substrate are detectable, and, after a certain time, the reaction is usually stopped. After the reaction has been stopped, reaction inhibition may be measured by determining the level of detectable reaction product present in the vessel. Reaction inhibition occurs when the rate of the reaction is reduced greater than about 20%, greater than about 50%, greater than about 70%, greater than about 80%, or greater than about 90% or 95% or more, as compared to a control assay that does not contain antibody.

In vivo Assays

In certain embodiments the monoclonal antibodies are tested in vivo. In general, the method involves administering a subject monoclonal antibody to an animal model for a disease or condition and determining the effect of the monoclonal antibody on the on the disease or condition of the model animal. In vivo assays of the invention include controls, where suitable controls include a sample in the absence of the monoclonal antibody. Generally a plurality of assay mixtures is run in parallel with different antibody concentrations to obtain a differential response to the various concentrations. Typically, one of these concentrations serves as a negative control, i.e. at zero concentration or below the level of detection. A monoclonal antibody of interest is one that modulates, i.e., reduces or increases a symptom of the animal model disease or condition by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the antibody. In general, a monoclonal antibody of interest will cause a subject animal to be more similar to an equivalent animal that is not suffering from the disease or condition. Monoclonal antibodies that have therapeutic value that have been identified using the methods and compositions of the invention are termed “therapeutic” antibodies.

Utility

These methods and compositions described herein have several uses, many of which will be described below.

In one embodiment, the invention provides methods of treating a subject with a monoclonal antibody of interest. In general these methods involve administering a monoclonal antibody identified by the methods described above to a host in need of treatment. In many embodiments, the monoclonal antibody is a therapeutic monoclonal antibody.

By treatment is meant at least an amelioration of a symptom associated with the pathological condition afflicting the host, where amelioration is used in a broad sense to refer to at least a reduction in the magnitude of a parameter, e.g. symptom, associated with the pathological condition being treated. As such, treatment also includes outcomes where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans. In other embodiment, the host will be an animal model for a human disease.

Of particular interest is treatment and prevention of diseases, conditions and disorders associated with abnormal expression of a cellular protein, usually present on the surface of a cell, e.g. a cancer cell.

The methods and compositions of the invention have several research applications. In one exemplary application, the library of monoclonal antibodies is deposited onto an array or microarray (e.g. using a method provided by U.S. Pat. Nos. 6,372,483, 6,352,842, 6,346,416 and 6,242,266), and labeled samples (e.g. cell extracts or proteins) or pairs of differentially labeled are incubated with the array. Such experiments may provide monoclonal antibodies and antibody-encoding polynucleotide sequences that differentially bind to samples. In one exemplary experiment, cancerous cells or extracts thereof are labeled and incubated with an array of monoclonal antibodies. After washing of the array, data representing the amount of binding of the cell or extract thereof may be extracted for each antibody. Comparison of this data to data generated using normal or non-cancerous cells incubated with a similar or the same array may reveal monoclonal antibodies that specifically recognize the cancer cell. Such antibodies have therapeutic applications.

The methods and compositions of the invention provide specific reagents that can be used in standard diagnostic procedures. For example, the antibodies or their immunoreactive fragments can be employed in immunoassays for detection of target antigens. To perform a diagnostic method, on of the compositions of the invention is provided as a reagent to detect a target antigen in a sample with which it reacts. Procedures for performing immunoassays are well established in the art and hence are not described here.

The monoclonal antibodies generated by the subject methods may also be used for treatment or prevention of diseases and conditions. The monoclonal antibodies may be used to modulate the activities of target antigens that play a central role in disease development and/or progression. For example, a humanized anti-Her2 antibody, available commercially under the trademark HERCEPTIN®, which selectively inhibits growth of human breast cancer cells, is now employed as a potent drug to treat tens and thousands of breast cancer patients who overekpress the breast cancer antigen Her2.

Kits

Also provided by the subject invention are kits for practicing the subject methods, as described above. The subject kits at least include one or more of: a library of mammalian cells displaying a plurality of different monoclonal antibodies, a library of mammalian cells displaying monoclonal antibodies, where each cell displays a different monoclonal antibody on its surface, a library of expression cassettes, for example linear or circular (e.g., in a retroviral vector) expression cassettes that provide for expression of a library of monoclonal antibodies in mammalian cells, or a parental monoclonal antibody. Other optional components of the kit include: components for performing antibody binding assays, e.g. FACS assays, microtiter plates and ELISA reagents; buffers, nucleotides and reagents for performing amplifying heavy and light chain nucleic acids; and a humanized antibody framework-encoding nucleic acids that is operably linked to a cell surface targeting sequence. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention is are kits including at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means for producing rabbit antibodies that are less immunogenic in a non-rabbit host than a parent antibody, or nucleotide sequences them.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Cloning of Rabbit Monoclonal Antibody B1

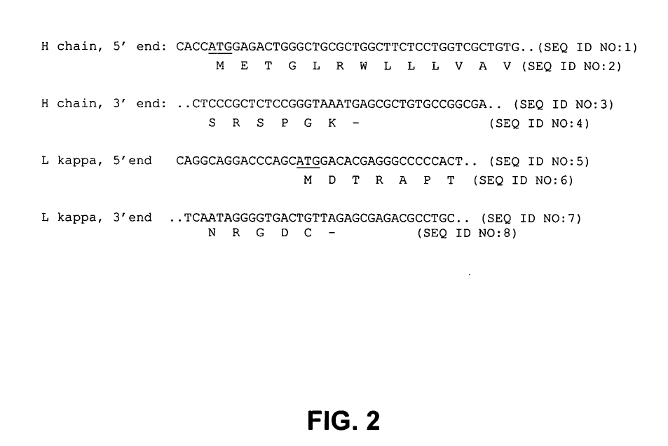

Compared to mouse IgG, the rabbit provides a major technical advantage for PCR amplification of heavy and light chain cDNAs. The mouse V_{H} and V_{L} genes have divergent sequences with respect to the 5′ ends of their coding regions. Therefore, a mixture of many different primers has to be used to amplify mouse antibody sequences. In contrast, the 5′ coding sequences of rabbit antibodies are primarily derived from only one gene. Thus, most rabbit IgG H chains have very similar or identical signal peptide sequences, and the same is the case with L chains. On the 3′ side, we use primers hybridizing to the constant domains, which also have identical sequences in most rabbit antibodies (rabbit constant domains are not divided into subclasses, e.g., IgG1, IgG2a, etc.). As a result, only one pair of primers each is required for amplifying the vast majority of rabbit IgG H and L sequences. Typical priming sites are shown in FIG. 2.

RT-PCR is performed using purified mRNA, or directly using the cell lysate. For direct RT-PCR of cell lysate, we will use a reagent kit specifically designed for high-throughput RT-PCR from small numbers of cells (cells-to-cDNA kit, Ambion, Austin Tex.). The procedure involves cell lysis by heating in a buffer containing RNAse inhibitors, followed by DNA degradation and reverse transcription performed at high temperatures (60° C.). Reverse transcription is primed either by oligo-dT or by primers specific for the 3′ region of the IgG mRNAs. We use a single-step RT-PCR protocol, utilizing a thermostable enzyme that has both reverse transcriptase and DNA polymerase activities (MasterAmp™ RT-PCR Kit for High Sensitivity, Epicentre Technologies, Madison, Wis.). PCR products are analyzed by agarose gel electrophoresis. If required, a second round of PCR will be performed with nested primers. In many PCR applications, this step is required to produce sufficient amounts of specific product. Partial sequencing of the PCR inserts is done using the PRISM Ready Reaction Cycle sequencing kit (ABI, Foster City, Calif.). The total sequence of isolated heavy and light chains of a monoclonal antibody obtained from hybridoma line B1 (Xue et al, Biochem Biophys Res Commun. 2001 288:610-8; Huang et al, Am J Respir Cell Mol Biol. 1998, 19:636-42), which binds the P6 integrin molecule that has been implicated to play a role in oral and colon cancer, is determined using primers designed after the initial sequencing.

### Example 2

### Humanization of Rabbit Monoclonal Antibody B 1 -CDR Grafting

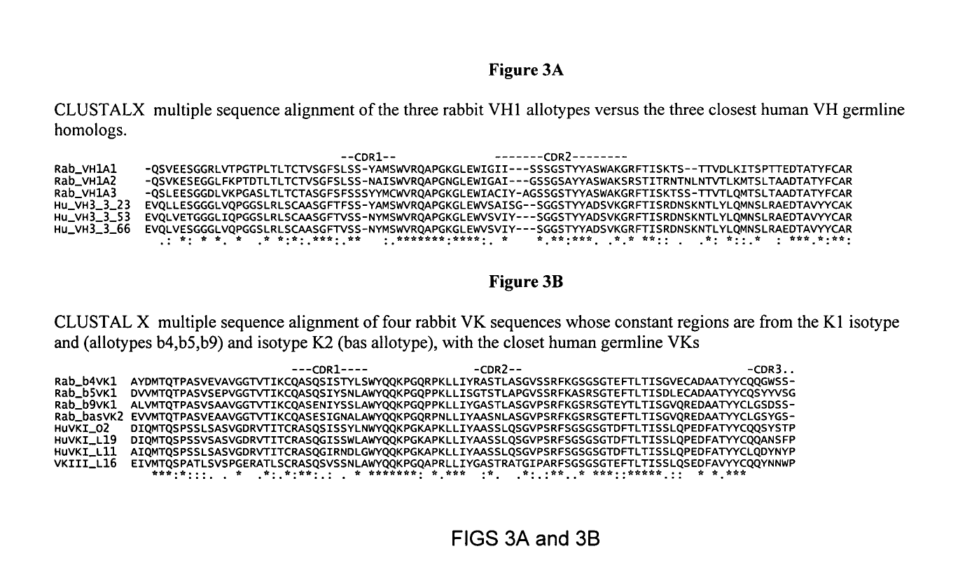

Analysis of the germline sequences of rabbit antibody genes provides valuable information on rabbit MAbs. Sequence comparison revealed that the rabbit antibody genes are similar to human antibody sequences. There are three major allotypes of rabbit V_{H}1 genes (a1, a2 and a3). As demonstrated by the sequence alignment shown in FIG. 3, rabbit V_{H}1 gene (a1 allotype) showed 62% identity to a human antibody germline sequence of Subgroup III, V3 3-23; 58% identities to V3 3-53. V_{H}1-a2 showed 57% identity to immunoglobulin heavy chain variable region of human V_{H}3 3-66 and 56% identity to VH3 3-53. V_{H}1-a3 showed up 65% identity to the human antibody heavy chain of V_{H}3-66; 65% identity to VH3 3-23; 63% identity to VH3 3-53. VL-kappa chain (b4VK1) showed 68% identity to human IgK subgroup I germline gene VKI O2; Rabbit b5VK1 has 60% identity to human VKIII L16; Rabbit b9VK1 has 68% identity to human VKI L19; Rabbit basVK2 has 67% identity to human VKI L11. Note that the sequence identity analysis include CDR regions of the genes. The framework regions have much higher homology to human sequences. The finding that rabbit antibody sequences can be readily aligned with human sequences shows that the rabbit antibodies may be altered without substantially changing the conformation of rabbit antibody molecules, and thus retain the affinities of the modified rabbit antibodies.

Once the sequence of the B1 antibody is available, various antibody gene databases are searched to identify the most homologous human antibody genes for B1, to provide the framework for CDR grafting. The rabbit B1 sequences are aligned with human V and J genes. In addition to National Center for Biotechnology Information (NCBI) databases, several of the most commonly used databases include: V BASE (Database of Human Antibody Genes) and Kabat Database of Sequences of Proteins of Immunological Interest and Immunogenetics Database. Built-in search engines can be used to find the most homologous amino acid sequences. One of the most commonly used program is BLAST (Basic Local Alignment Search Tool).

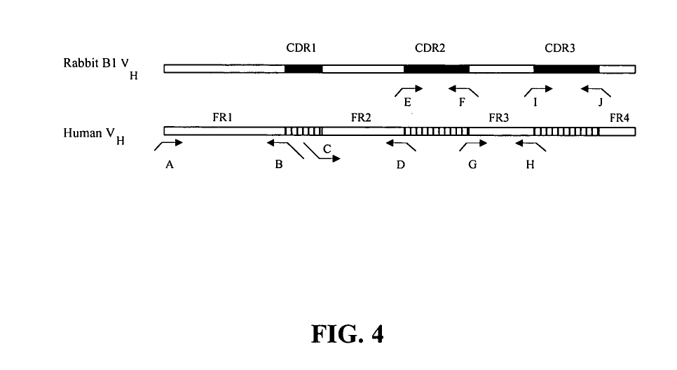

Overlapping PCR are used to create the chimeric structure of a humanized VH and VL. An example of overlapping PCR method is described in FIG. 4. Briefly, primer pairs A+B and C+D are used to amplify the FR1 and FR2 region of human VH. The mutations necessary to change the human VH CDR1 sequence to that of the rabbit CDR1 can be designed into primers B and C. The rabbit VH CDR2 is amplified with primers E+F, the human VH FR3 with primers G+H and the rabbit VH CDR3 with primers I+J. A humanized JH-region/FR4 can be encoded partially by primer J, partially by vector. The PCR products resulting from these PCR reactions are purified, combined and subjected to 10 cycles of PCR amplificaton without primers. Primers A+J are then added and a standard 25 cycle PCR protocol will be performed. A similar strategy can be applied to CDR-grafting of VL. The resulting PCR product is cloned to E.Coli or mammalian expression vector. In the case of E. coli expression, a soluble Fab fragment is preferred. The Fab fragment, preferably tagged, will be released from the periplasm and purified by affinity chromatography. For mammalian cell expression, transient transfection of light and heavy chain genes will produce secreted Fab or full-length antibody molecules. As an alternative method, the V_{H} and V_{L} region of the CDR-grafted DNA can be synthesized by commercial sources (whole-gene synthesis). The synthesized genes, containing the sequences of humanized heavy- and light-chain variable region, will be cloned to expression vectors to express Fab fragments or full-length antibodies.

Previous reports have demonstrated the importance of framework residues of mouse MAb in maintaining affinity. For example, two amino acid residues from the murine sequence immediately before CDR1 and one before CDR2 are relatively more likely to be non-solvent-exposed ‘packing’ residues than amino acids at other positions. Residues at similar positions in rabbit antibodies may be subjected to ‘back-mutation’ by site-directed mutagenesis. Molecular modeling of rabbit antibody sequences, using programs such as ABMOD and ENCAD, may be used to determine the residues located close to CDRs, which may need to be preserved as rabbit residues in the humanized molecules. Commercial kits are available for high efficiency mutagenesis. Quickchange mutagenesis kit (Stratagene, La Jolla, Calif.) is used for this experiment.

CDR-grafted heavy and light chain genes with and without back-mutations is subcloned into mammalian cell expression vector pcDNA3.1 (Invitrogen, Carlsbad, Calif.). Humanized heavy and light chains are co-expressed in CHO cells. The supernatants from the transiently transfected cells are used in ELISA assays to determine the binding of antibodies to β6 integrin. Affinity of the positive binding MAbs are analyzed by BIAcore instruments (Pharmacia, Peapack, N.J.). B1 rabbit MAb is used as positive control in these binding experiments. It is expected that the affinity of the humanized B1 may decrease due to the interaction of human framework with rabbit CDRs. However, back-mutations may partially or fully restore the binding affinity without causing significant immunogenecity to humans.

### Example 3

### Humanization of Rabbit Monoclonal Antibody B1 -Resurfacing

To ‘resurface’ the antibody sequence, the sequences of the variable domains of the rabbit MAb and of the selected frameworks are compared, and surface framework residues from the rabbit antibody are substituted with the human residues. The x-ray crystallographic structures were used to determine distributions of surface exposed amino acid residues (Padlan et al, Mol Immunol. 1991 28:489-98; Roguska et al, Proc Natl Acad Sci 1994 91:969-73; Pedersen et al, J Mol Biol. 1994 235:959-73). The same type of modeling is applied to newly discovered structures that are updated in various databases. The relative accessibility distributions with residues of 30% or over-is calculated using a method of DSSP (Dictionary of Secondary Structure in Proteins) by Kabsch and Sander (Kabsch and Sander Biopolymers. 1983 22:2577-637). The result can are then used to analyze the human genes most homologous to specific rabbit antibody sequences (heavy chain and light chain). After the surface residues for the most homologous human antibody genes are determined, sequence alignment of these genes to rabbit antibody gene in question is performed. Residues at corresponding positions on the rabbit gene are predicted to be most likely surface residues. Due to the similarity of rabbit and human antibody sequences in framework regions, the alignment accurately identifies the set of possible framework positions of surface exposed residues on rabbit antibodies.

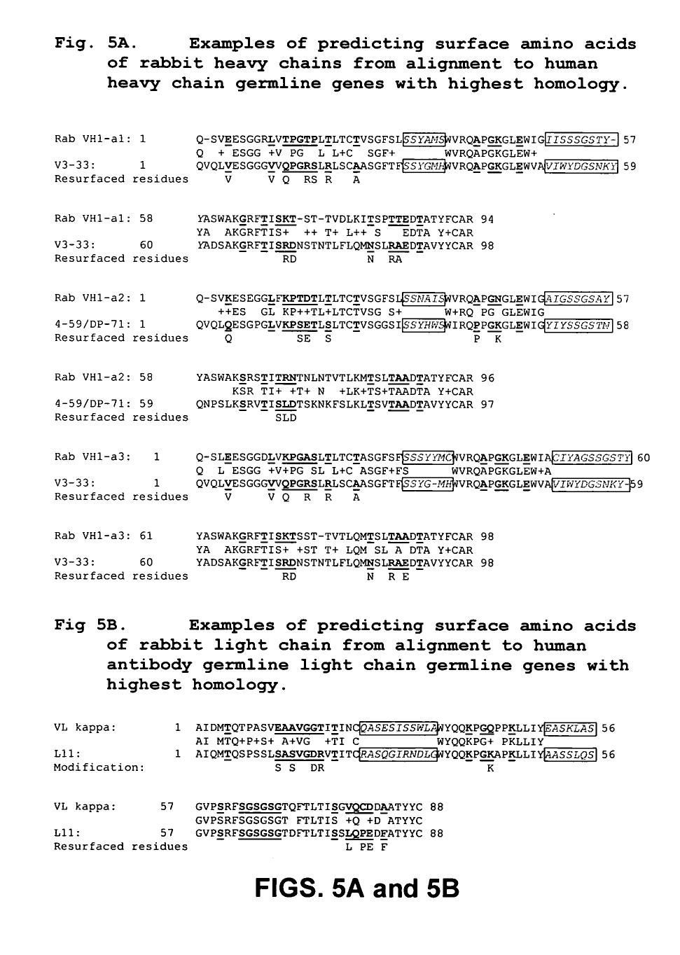

As a more direct approach, the result from previous studies using limited crystallographic antibody structures are used directly to analyze rabbit sequences using protein alignment programs. Since human and mouse surface residues are predicted from the previous studies, the possible rabbit surface residues are deduced from sequence alignment analysis. Examples of humanization of rabbit antibodies using rabbit germline genes are shown below in FIG. 5 as panel A and B. In A, framework sequences of V_{H}1 (germline) genes of a1, a2, a3 allotypes are aligned with most homologous human germline antibody genes from NCBI and V Base databases. Specifically, V3-33 is used to align with V_{H}1-a1 and V_{H}1-a3, 4-59/DP-71 is used to align with V_{H}1-a2. The surface residues in FR1, FR2 and FR3 of the most homologous human antibodies are predicted and highlighted. From the sequence alignment result, rabbit framework residues region at homologous positions are identified as most likely to be accessible to solvent, and are shaded in the figure. The same process was used to analyze V_{L} frame from rabbit Ig germline kappa (b4) V-region gene V19b (shown in B). In this example, 2-1-(1), L11 is used to align with rabbit V19b. The residues to be resurfaced are shown at the bottom of each alignment.

A more precise method to predict surface residues in rabbit antibodies is through construction of three-dimensional models for the original and humanized antibodies. This is done by known methods for modeling murine and human antibodies, described by known methods. This method, called Combined Algorithm for Modeling Antibody Loops (CAMEL), is able to predict the backbone conformations of all six CDRs of the antibody binding site, as well as fitting together the framework regions. This method applies to human and murine antibodies equally well. Due to the similarity of antibody structures between rabbit and human/mouse, it is expected that the same algorithm will work for rabbit antibodies.

### Example 4

### Mammalian Cell Display

Linear DNA:

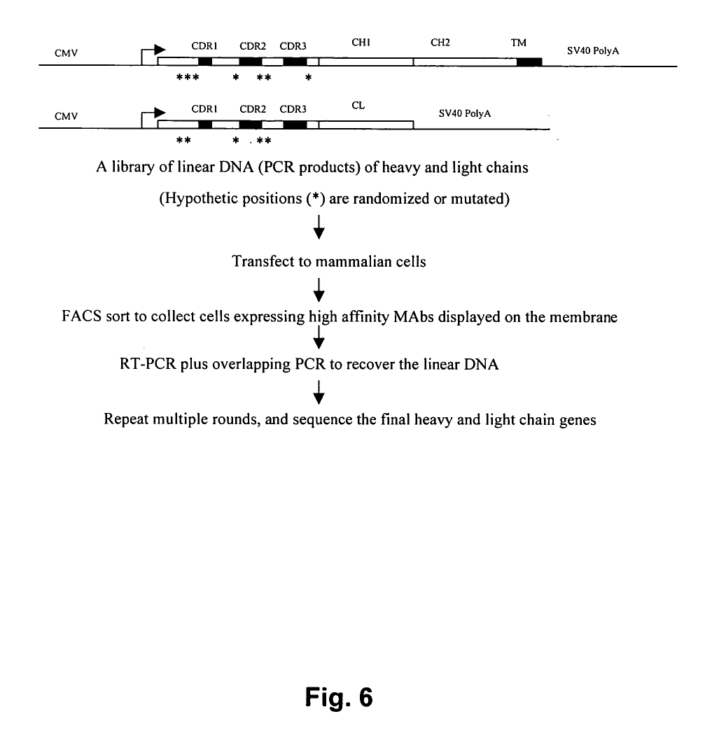

In this method, antibody heavy chain and light chain mutant libraries are constructed as linear DNA by overlapping PCR to contain a promoter and a polyadenylation sequence, so that it can be transcribed in mammalian cells. CDRs of B1 are grafted onto human framework as described in FIG. 6. At the same time, either rabbit or human residues are introduced to designated ‘back-mutation’ positions by randomly incorporation of desired nucleotides during oligonucleotide synthesis. During the process of overlapping PCR, such mutations are incorporated into the linear DNA with random combinations. For example, a position to be mutated can be either a human residue or a rabbit residue. Such linear DNA will contain a mammalian cell promoter (such as CMV promoter), the full-length antibody heavy or light chain gene, a transmembrane (TM) domain of a known protein (such as PDGF receptor TM domain) fused in-frame with the heavy chain, and a poly-adenylation sequence (such as SV40). From our previous experience, such linear DNA can express functional antibodies from transiently transfected 293 cells (data not shown). The heavy and light chain linear DNA libraries are transfected transiently into mammalian expression cell lines such as 293 cells or Cos-7 cells. After 24-48 hr, the cells are sorted by flow cytometry. Fluorescently labeled P6 antigen may be used for FACS sorting, and use unlabeled antigen for competitive binding to select MAbs of higher affinity. The cells expressing the most abundant MAbs with highest-binding affinities are isolated. The genes encoding heavy and light chains are recovered from the isolated cells by RT-PCR. After adding the promoter and poly(A)ₙ segments at the 5′ and 3′ ends of the antibody genes respectively by overlapping PCR, the linear DNA sub-libraries are used to reiterate the above process, and the functional antibody genes are enriched. The linear DNA can be recovered by direct PCR using primers for both ends of the DNA.

The fraction of cells expressing the desired antibodies should increase with each round of enrichment. The antibody genes will be cloned into plasmid vectors. Individual plasmid of heavy and light chain genes will be paired, tested for expression of functional antibodies, and subjected to DNA sequencing. From this process, the optimal combinations of back-mutants are obtained without the need of individual site-directed mutagenesis. It may not be known how many copies of these linear DNA molecules are expressed in each cell. Nevertheless, the process is likely to promote heavy/light chain shuffling (when more than one pairs of antibody genes are present in the same cells) and the selection of cells expressing higher copy numbers of desired antibody genes. The concept may be amenable to antibody maturation, in which case selected residues in CDRs (especially CDR3) can be randomized. The diagram of this procedure is shown in FIG. 6.

Retroviral Vectors

Retroviral gene transfer is a technique for efficiently introducing stable, heritable genetic material into the genome of any dividing cell type. Recent advances in viral packaging systems allow virtually any mitotic cell type to be transduced with efficiencies approaching 100%. The copy number of individual cDNA expression cassettes can be easily controlled by varying the multiplicity of infection (MOI). Thus, populations of infected cells may be generated in which greater than 90% of the cells are transduced with 1-5 individual cDNAs per cell, greatly reducing the time and labor of isolating the gene of interest.

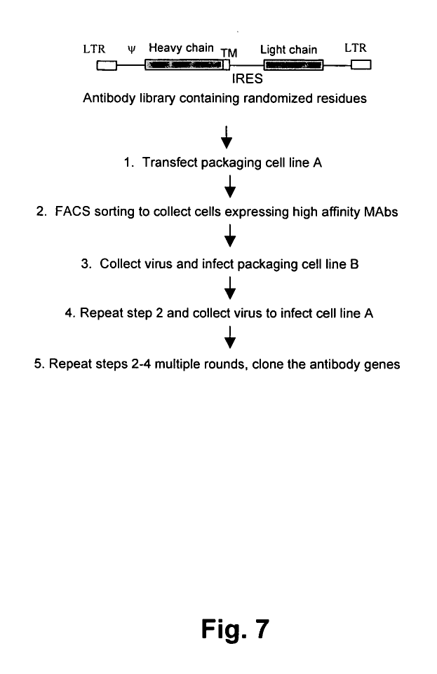

The antibody heavy and light chain libraries is constructed in a single retroviral vector with internal ribosomal entry site (IRES) engineered in between the heavy and light chain genes, so that both genes can be translated. The retroviral expression library is transfected to a packaging cell line (such as EcoPack-293, Clontech). The cells will express antibody molecules that are anchored on the cell membrane by exogenous transmembrane domain (such as PDGF receptor TM). The cells that express the desired antibodies are collected by FACS sorting. The virus from the culture supernatant of the sorted cells will be used to infect a different packaging cell line (such as RetroPack PT67). Because these two cell lines express different envelope proteins, one cell line can get transduced with the virus from the other cell line. After FACS sorting the second packaging cells for high affinity surface antibody expression, the virus from the sorted cells will be used to infect the first packaging line. After multiple rounds, the final antibody expressing-cells will be subjected to RT-PCR to recover the antibody genes. The advantage of using two packaging cell lines for mutual infection include: 1) increased infection efficiency, and 2) heavy/light chain shuffling when more than one viral vectors enter the same packaging cell. The procedure is shown in FIG. 7.

It is evident from the above results and discussion that the subject invention provides an important new means for generating monoclonal antibodies with improved activity. Specifically, the subject invention provides a library of variant monoclonal antibodies that are derived from a single parental monoclonal antibody, and methods for screening that library to identify a monoclonal antibody of interest with an improved activity. As such, the subject methods and systems find use in a variety of different applications, including research, therapeutic and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for producing a library of monoclonal antibodies, said method comprising:
a) altering a nucleic acid encoding a selected humanized monoclonal antibody to provide a library of humanized monoclonal antibody-encoding nucleic acids; and
b) introducing said library into mammalian cells to produce monoclonal antibodies on the surfaces of said cells to obtain said library of monoclonal antibodies.

2. The method of claim 1, wherein said monoclonal antibody is a rabbit monoclonal antibody.

3. The method of claim 1, wherein said monoclonal antibody is humanized by CDR grafting or resurfacing.

4. The method of claim 1, wherein said altering is making random alterations.

5. The method of claim 4, wherein said random alterations are distributed within nucleic acids encoding a CDR or variable domain framework region of an immunoglobulin heavy or light chain for said antibody.

6. The method of claim 4, wherein said random alterations are at a position corresponding to a single amino acid within a variable domain framework region of an immunoglobulin heavy or light chain for said antibody.

7. The method of claim 1, wherein said altering is making directed changes.

8. The method of claim 7, wherein said alterations change one amino acid into one of a number of pre-determined amino acids.

9. The method of claim 1, wherein said library is a library of linear expression cassette pairs that provide for immunoglobulin heavy and light chain expression and said introducing step is introducing said linear expression cassette pairs into said cells.

10. The method of claim 1, wherein said library is a library of retroviral vectors that provide for immunoglobulin heavy and light chain expression and said introducing step is introducing said retroviral vectors into said cells.

11. The method of claim 10, wherein said retroviral vector comprises a light chain expression cassette and a heavy chain expression cassette separated by an internal ribosome entry site (IRES).

12. A method for identifying a cell producing a first monoclonal antibody of interest, said method comprising:
producing a library of cell surface expressed humanized monoclonal antibodies according to the method set forth in claim 1I; and
screening said antibodies to identify a cell producing a first monoclonal antibody of interest.

13. The method of claim 12, wherein said screening is by separating cells producing said first monoclonal antibody of interest from other cells.

14. The method of claim 12, wherein said screening is by affinity of specificity to an antigen.

15. The method of claim 14, wherein said selecting cells is by FACS sorting.

16. The method of claim 1, wherein said introducing step is introducing said library into mammalian cells to produce monoclonal antibodies on the surfaces of said cells to obtain said library of monoclonal antibodies, wherein each cells produce a plurality of different antibodies.

17. A method for identifying a cell producing a monoclonal antibody of interest, said method comprising:
(a) producing a library of cell surface expressed monoclonal antibodies according to the method set forth in claim 1;
(b) isolating cells producing said monoclonal antibody of interest by their binding to an antigen;
(c) obtaining nucleic acid encoding said monoclonal antibody of interest from said isolated cells;
(d) introducing said nucleic acid into mammalian cells to produce a second library of cell surface expressed monoclonal antibodies; and
(e) screening said library to identify a cell producing a monoclonal antibody of interest.

18. The method of claim 17, wherein said library is in a retroviral vector and said mammalian cells used in said producing step (a) and said mammalian cells used in said introducing step (d) are different retroviral packaging cell lines.

19. The method of claim 17, wherein said library is a library of linear expression vectors.

20. A method for identifying a second monoclonal antibody of interest, said method comprising:
a) altering a nucleic acid encoding a first monoclonal antibody of interest identified by the method of claim 10 to provide a library of humanized monoclonal antibody-encoding nucleic acids;
b) introducing said library into mammalian cells to produce a library of cell surface expressed humanized monoclonal antibodies; and
screening said antibodies to identify a monoclonal antibody of interest.


SEQUENCE LISTING (38 sequences)

SEQ ID NO: 1 - DNA, 43 bp, Oryctolagus cuniculus
caccatggagactgggctgcgctggcttctcctggtcgctgtg

SEQ ID NO: 2 - protein, 13 aa, Oryctolagus cuniculus
METGLRWLLLVAV

SEQ ID NO: 3 - DNA, 37 bp, Oryctolagus cuniculus
ctcccgctctccgggtaaatgagcgctgtgccggcga

SEQ ID NO: 4 - protein, 6 aa, Oryctolagus cuniculus
SRSPGK

SEQ ID NO: 5 - DNA, 36 bp, Oryctolagus cuniculus
caggcaggacccagcatggacacgagggcccccact

SEQ ID NO: 6 - protein, 7 aa, Oryctolagus cuniculus
MDTRAPT

SEQ ID NO: 7 - DNA, 34 bp, Oryctolagus cuniculus
tcaataggggtgactgttagagcgagacgcctgc

SEQ ID NO: 8 - protein, 5 aa, Oryctolagus cuniculus
NRGDC

SEQ ID NO: 9 - protein, 94 aa, Oryctolagus cuniculus
QSVEESGGRLVTPGTPLTLTCTVSGFSLSSYAMSWVRQAPGKGLEWIGIISSSGSTYYASWAKGRFTISKTSTTVDLKITSPTTEDTATYFCAR

SEQ ID NO: 10 - protein, 96 aa, Oryctolagus cuniculus
QSVKESEGGLFKPTDTLTLTCTVSGFSLSSNAISWVRQAPGNGLEWIGAIGSSGSAYYASWAKSRSTITRNTNLNTVTLKMTSLTAADTATYFCAR

SEQ ID NO: 11 - protein, 98 aa, Oryctolagus cuniculus
QSLEESGGDLVKPGASLTLTCTASGFSFSSSYYMCWVRQAPGKGLEWIACIYAGSSGSTYYASWAKGRFTISKTSSTTVTLQMTSLTAADTATYFCAR

SEQ ID NO: 12 - protein, 98 aa, Oryctolagus cuniculus
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 13 - protein, 97 aa, Oryctolagus cuniculus
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 14 - protein, 97 aa, Oryctolagus cuniculus
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 15 - protein, 94 aa, Oryctolagus cuniculus
AYDMTQTPASVEVAVGGTVTIKCQASQSISTYLSWYQQKPGQRPKLLIYRASTLASGVSSRFKGSGSGTEFTLTISGVECADAATYYCQQGWSS

SEQ ID NO: 16 - protein, 95 aa, Oryctolagus cuniculus
DVVMTQTPASVSEPVGGTVTIKCQASQSIYSNLAWYQQKPGQPPKLLISGTSTLAPGVSSRFKASRSGTEFTLTISDLECADAATYYCQSYYVSG

SEQ ID NO: 17 - protein, 94 aa, Oryctolagus cuniculus
ALVMTQTPASVSAAVGGTVTIKCQASENIYSSLAWYQQKPGQPPKLLIYGASTLASGVPSRFKGSRSGTEYTLTISGVQREDAATYYCLGSDSS

SEQ ID NO: 18 - protein, 94 aa, Oryctolagus cuniculus
EVVMTQTPASVEAAVGGTVTIKCQASESIGNALAWYQQKPGQRPNLLIYAASNLASGVPSRFKGSRSGTEFTLTISGVQREDAATYYCLGSYGS

SEQ ID NO: 19 - protein, 95 aa, Oryctolagus cuniculus
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 20 - protein, 95 aa, Oryctolagus cuniculus
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 21 - protein, 95 aa, Oryctolagus cuniculus
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 22 - protein, 95 aa, Oryctolagus cuniculus
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 23 - protein, 94 aa, Oryctolagus cuniculus; identical to SEQ ID NO: 9

SEQ ID NO: 24 - protein, 98 aa, Oryctolagus cuniculus
QVQLVESGGGVVQPGRSLRLSCAASGFTFSSYGMHWVRQAPGKGLEWVAVIWYDGSNKYYADSAKGRFTISRDNSTNTLFLQMNSLRAEDTAVYYCAR

SEQ ID NO: 25 - protein, 96 aa, Oryctolagus cuniculus; identical to SEQ ID NO: 10

SEQ ID NO: 26 - protein, 97 aa, Oryctolagus cuniculus
QVQLQESGPGLVKPSETLSLTCTVSGGSISSYHWSWIRQPPGKGLEWIGYIYSSGSTNQNPSLKSRVTISLDTSKNKFSLKLTSVTAADTAVYYCAR

SEQ ID NO: 27 - protein, 98 aa, Oryctolagus cuniculus; identical to SEQ ID NO: 11

SEQ ID NO: 28 - protein, 88 aa, Oryctolagus cuniculus
AIDMTQTPASVEAAVGGTITINCQASESISSWLAWYQQKPGQPPKLLIYEASKLASGVPSRFSGSGSGTQFTLTISGVQCDDAATYYC

SEQ ID NO: 29 - protein, 88 aa, Oryctolagus cuniculus
AIQMTQSPSSLSASVGDRVTITCRASQGIRNDLGWYQQKPGKAPKLLIYAASSLQSGVPSRFSGSGSGTDFTLTISSLQPEDFATYYC

SEQ ID NO: 30 - DNA, 43 bp, Oryctolagus cuniculus; identical to SEQ ID NO: 1

SEQ ID NO: 31 - DNA, 37 bp, Oryctolagus cuniculus; identical to SEQ ID NO: 3

SEQ ID NO: 32 - DNA, 36 bp, Oryctolagus cuniculus; identical to SEQ ID NO: 5

SEQ ID NO: 33 - DNA, 34 bp, Oryctolagus cuniculus; identical to SEQ ID NO: 7

SEQ ID NO: 34 - protein, 31 aa, Oryctolagus cuniculus
MASPLTRFLSLNLLLLGESILGSGEAKPQAP

SEQ ID NO: 35 - protein, 21 aa, Oryctolagus cuniculus
MSSFGYRTLTVALFTLICCPG

SEQ ID NO: 36 - protein, 51 aa, Oryctolagus cuniculus
PQRPEDCRPRGSVKGTGLDFACDIYIWAPLAGICVALLLSLIITLICYHSR

SEQ ID NO: 37 - protein, 33 aa, Oryctolagus cuniculus
MVIIVTVVSVLLSLFVTSVLLCFIFGQHLRQQR

SEQ ID NO: 38 - protein, 37 aa, Oryctolagus cuniculus
PNKGSGTTSGTTRLLSGHTCFTLTGLLGTLVTMGLLT